(19) United States
(12) Patent Application Publication
(10) Pub. No.: US 2019/0343590 A1
(43) Pub. Date: Nov. 14, 2019

(54) Systems and Methods for a Template System Used In Image Guided Medical Procedures

(71) Applicant: The Brigham and Women's Hospital, Inc., Boston, MA (US)

(72) Inventors: Nobuhiko Hata, Waban, MA (US); Petyer Tia, Dracut, MA (US)

(21) Appl. No.: 15/755,029

(22) PCT Filed: Aug. 26, 2016
(86) PCT No.: PCT/US16/48897

§ 371 Date: Feb. 23, 2018

Related U.S. Application Data
(60) Provisional application No. 62/211,035, filed on Aug. 28, 2015.
(60) Provisional application No. 62/276,152, filed on Jan. 7, 2016.

(51) Int. Cl. A61B 34/20; A61B 17/34; A61B 34/32

(52) U.S. Cl. CPC A61B 34/20; A61B 17/3403; A61B 34/32; A61B 2090/374; A61B 2017/3411; A61B 2034/2051; A61B 2034/2065; A61B 2017/3409

(57) ABSTRACT

The present disclosure provides systems and methods for a template system for an image guided medical procedure. In particular, systems and methods are provided for a template system to create holes in a template for guiding a needle with the assistance of medical imaging system such as Magnetic Resonance Imaging. The template is used to guide one or more needles to an area of interest in a medical procedure.

### CROSS-REFERENCES TO RELATED APPLICATIONS

This application is based on, claims priority to, and incorporates herein by reference in its entirety, each of U.S. Provisional Application Ser. No. 62/211,035, filed Aug. 28, 2015, and entitled and entitled “NEEDLE PLACEMENT GUIDANCE TEMPLATES,” and U.S. Provisional Application Ser. No. 62/276,152, filed Jan. 7, 2016, and entitled “SYSTEMS AND METHODS FOR A TEMPLATE SYSTEM USED IN IMAGE GUIDED MEDICAL PROCEDURES.”

### STATEMENT REGARDING FEDERALLY SPONSORED RESEARCH

This invention was made with government support under grant no. P41EB015898/EB awarded by the National Institutes of Health. The government has certain rights in the invention.

### BACKGROUND

This disclosure relates generally to medical procedures and, more specifically, to template systems and methods using the same for image-guided medical procedures.

Targeted procedures to aim suspicious foci visible in Magnetic Resonance Imaging (MRI) have been proposed as a strong alternative to other systematic non-targeted procedures. MRI provides improved imaging characteristics and an increased ability to define suspicious areas and detect, characterize, and stage potentially harmful tissue (e.g., cancerous tissue). Additionally, MRI provides superior visualization of suspicious foci and can both target and guide treatment and diagnostic apparatus to the foci enabling image targeted/guided procedures.

Examples of procedures of interest in this field include but are not limited to biopsies, ablations, or brachytherapy. These procedures can be performed under ultrasound, X-ray Computed Tomography (CT), or MRI guidance. In case of ultrasound or CT-guided procedures, the MRI taken before the procedures but also used to define the foci, will be digitally fused to intra-operative ultrasound or CT. This image fusion helps physicians to localize foci in the ultrasound or CT, which otherwise cannot localize the foci. In case of MRI-guided procedures, MRI taken before the procedure but also used to define the foci, can be also fused to intra-operative MRI to identify target foci in the intra-operative MRI. One can also use intra-operative MRI only to set foci to aim in the procedures.

Currently, the foci can be aimed by diagnostic and therapeutic tools using a perforated template tool guide with a pre-defined tool-guide holes having discrete spacing between holes. This discrete spacing between holes a template limits the accuracy of tool placement in targeted approaches, since MRI can localize suspicious foci in higher resolution than pre-defined hole pattern can handle. Additionally, clinician error can also occur because the clinician is responsible for counting small diameter holes in order to correctly place needles.

Therefore, there is a need to make an improved template system that can aim the foci with targeting resolution close to that of MRI.

### BRIEF SUMMARY

The present disclosure provides systems and methods for using a template system for an image-guided medical procedure. In particular, systems and methods are provided for a system to create a patient specific template for guiding a diagnostic and therapeutic tools with the assistance of MRI. The template is used to guide one or more tools to an area of interest in a medical procedure.

In one aspect, the present disclosure provides a method for creating a template using a robotic assembly of a template system. The template system including a template holder configured to receive the template, a guide coupling aperture, and a controller in communication with an imaging system. The method includes arranging the template holder adjacent to a region of interest of a patient such that the template holder restricts access to the region of interest of the patient, acquiring one or more images of the template holder and the region of interest of the patient with the imaging system, and determining, using the one or more images, an geometric location of the region of interest. The method further includes engaging the template with the robotic assembly of the robotic-guided assembly system, robotically guiding the guide coupling aperture to a location on the template corresponding to the geometric location of the region of interest, and creating a hole in the template at the location on the template corresponding to the geometric location of the region of interest.

In another aspect, the present disclosure provides a method of performing an image guide medical procedure on a patient within an imaging system. The method includes placing a template holder adjacent to a region of interest of the patient such that the template holder restricts access to the region of interest of the patient, and robotically creating a template. The template is robotically created by capturing one or more images of the template holder and the region of interest of the patient with the imaging system, identifying, using the one or more images, an area of tissue within the region of interest of the patient, engaging the template with a robotic assembly of a robotic-guided assembly system, robotically guiding a guide coupling aperture of the robotic assembly to a location on the template corresponding to the geometric location of the identified area of tissue, and creating a hole in the template at the location on the template corresponding to the geometric location of the identified area of tissue. The method further includes upon creating the hole in the template, disengaging the template from the robotic assembly and engaging the template with the template holder, inserting a needle through the hole in the template and into the patient, verifying a position of the needle within the patient via the imaging system, performing a treatment on the identified area of tissue within the patient, and removing the needle from the patient.

In yet another aspect, the present disclosure provides a method for automated guidance of an image-guided medical procedure. The method includes accessing one or more images of a region of interest of a patient and a template holder arranged adjacent to the region of interest of the patient such that the template holder restricts access to the region of interest of the patient from at least one direction, and using the one or more images of the region of interest and template holder, identifying desired tissue in the region of interest and a geometric location relative to the template holder that provides access to the desired tissue through the template holder from the at least one direction. The method further includes communicating the geometric location to a controller configured to create a hole in a template configured to be coupled with the template holder to provide access to the desired tissue from the at least one direction through the template when the template is engaged with the template holder, accessing one or more images of a needle being inserted through the hole in the template from the at least one direction and into the patient toward the desired tissue, and verifying a position of the needle within the patient using the one or more images of the needle being inserted.

In still another aspect, the present disclosure provides a template system for an image guided medical procedure performed on a patient. The template system includes a first motor, a second motor and a robotic assembly. The robotic assembly includes a robotic assembly frame including a first guide and a second guide. The first guide is moveable along a first axis and operably coupled to the first motor, and the second guide is moveable along a second axis substantially perpendicular to the first axis and operably coupled to the first motor. The robotic assembly further includes a hole guide coupling to couple the first guide to the second guide and having a guide coupling aperture. The template system further includes a template configured to be received within the robotic assembly frame, and a controller configured to control the first motor and the second motor to move the first guide and the second guide and thereby position the guide coupling aperture in one or more pre-determined locations.

In yet another aspect, the present disclosure provides a disposable template for a guided medical procedure. The template configured to be removably received within a template holder. The template includes a first side and a second side each coated with a removable thin film. The template is fabricated from a non-ferrous material.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF DRAWINGS

The invention will be better understood and features, aspects and advantages other than those set forth above will become apparent when consideration is given to the following detailed description thereof. Such detailed description makes reference to the following drawings.

FIG. 1 shows a schematic of a template system according to one embodiment of the present disclosure.

FIG. 2 shows an orthographic view of a template system according to one embodiment of the present disclosure.

FIG. 3 shows an exploded view of the template system of FIG. 2.

FIG. 4 shows a cross-sectional view of the template system of FIG. 2 taken along line 4-4.

FIG. 5 shows a cross-sectional view of the template system of FIG. 2 taken along line 5-5.

FIG. 6 shows an orthographic view of a template holder with a template in accordance with one embodiment of the present disclosure.

FIG. 7 shows a side view of the template holder with a template of FIG. 6.

FIG. 8 shows an MRI system in accordance with one embodiment of the present disclosure.

FIG. 9 shows a front view of a template holder and template in relation to a patient within an MR bore in accordance with one embodiment of the present disclosure.

FIG. 10 is a flow chart setting forth the pre-procedure steps for use of a template system in accordance with one embodiment of the present disclosure.

FIG. 11 is a flow chart setting forth the procedure steps for use of a template system in a non-limiting example of a biopsy procedure in accordance with one embodiment of the present disclosure.

FIG. 12 is a flow chart setting forth the procedure steps for use of a template system in a non-limiting example of an ablation or brachytherapy treatment procedure in accordance with one embodiment of the present disclosure.

### DETAILED DESCRIPTION

Robotic devices have been introduced to overcome some of the access challenges for magnetic resonance imaging (MRI)-guided procedures, and to improve tool placement accuracy with a goal of better diagnostic and therapeutic outcome. However, these current robotic devices are costly because they must be fabricated from a MRI compatible material. That is, current robotic devices are required to be placed within the limited spaced of an MR bore and, therefore, the entirety of the robotic device must be fabricated from a costly non-ferrous material. Additionally, current robotic devices are typically limited to holding one needle which limits the robotic device to only make multiple needle insertions one at a time (i.e., not simultaneously).

Due to the current deficiencies in robotic MRI-guided procedures, it would be desirable to have a template system for MRI-guided medical procedures having a disposable template for accurately locating a target area. The disposable template can improve targeting accuracy, acquisition of harmful tissue, and reduce procedure time as related to the manual procedures. Further, only the disposable template is required to be fabricated from an MRI compatible material which significantly reduces a cost of the template system when compared to current robotic devices.

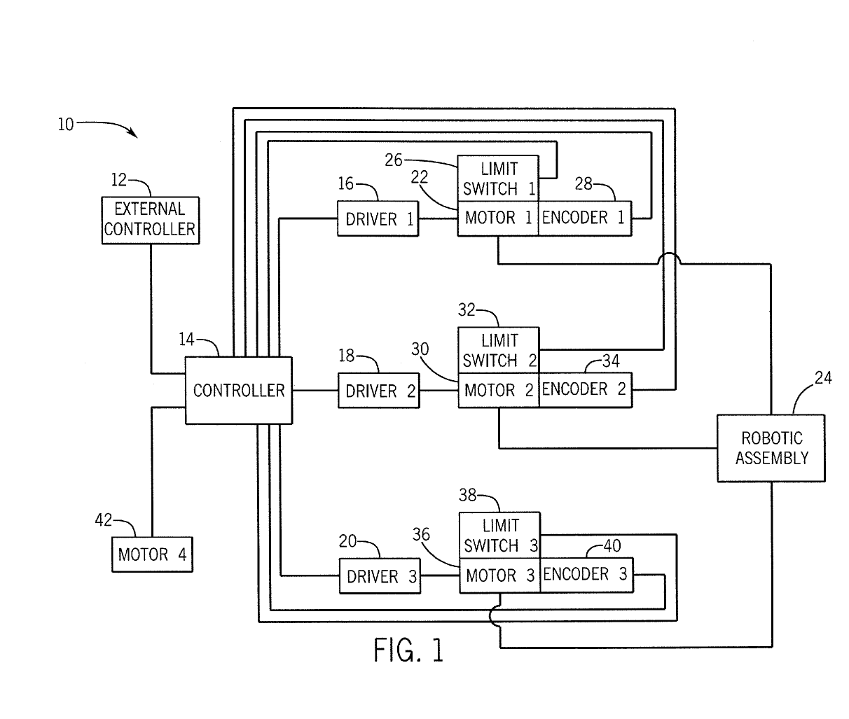

FIG. 1 shows one non-limiting example of a schematic of a template system 10 according to the present disclosure. The template system includes an external controller 12 in communication with a controller 14. The external controller 12 may be in direct wired communication (e.g., via a universal serial bus (USB) connection) with the controller 14, or the external controller 12 may be in wireless communication (e.g., via Bluetooth®, WiFi, etc.) with the controller 14. The controller 14 is connected to and configured to control a first driver 16, a second driver 18, and a third driver 20. The controller 14 sends signals to the first driver 16, the second driver 18, and the third driver 20 based on the data transferred from the external controller 12, as will be described in detail below.

The first driver 16 is connected to a first motor 22. The first motor 22 is coupled to a robotic assembly 24 and configured to provide motion of the robotic assembly 24 in along a first axis in response to a signal from the first driver 16. A first limit switch 26 is operably coupled to the first motor 22 and in communication with the controller 14. The first limit switch 35 is configured to prevent the first motor 22 from moving the robotic assembly 24 along the first axis beyond a pre-defined limit. A first encoder 28 is in communication with the controller 14 and configured to send positional feedback signals to the controller 14. That is, the first encoder 28 is configured to communicate a position of the robotic assembly 24 along the first axis to the controller 14.

The second driver 18 is connected to a second motor 30. The second motor 30 is coupled to the robotic assembly 24 and configured to provide motion of the robotic assembly 24 along a second axis perpendicular to the first axis in response to a signal from the second driver 18. A second limit switch 32 is operably coupled to the second motor 30 and in communication with the controller 14. The second limit switch 32 is configured to prevent the second motor 30 from moving the robotic assembly 24 along the second axis beyond a pre-defined limit. A second encoder 34 is in communication with the controller 14 and configured to send positional feedback signals to the controller 14. That is, the second encoder 34 is configured to communicate a position of the robotic assembly 24 along the second axis to the controller 14.

The third driver 20 is connected to a third motor 36. The third motor 36 is coupled to the robotic assembly 24 and configured to provide motion of the robotic assembly 24 along a third axis perpendicular to the first axis and the second axis in response to a signal from the third driver 20. A third limit switch 38 is operably coupled to the third motor 36 and in communication with the controller 14. The third limit switch 38 is configured to prevent the third motor 36 from moving the robotic assembly 24 along the third axis beyond a pre-defined limit. A third encoder 40 is in communication with the controller 14 and configured to send positional feedback signals to the controller 14. That is, the third encoder 40 is configured to communicate a position of the robotic assembly 24 along the second axis to the controller 14. A fourth motor 42 is connected to the controller 14 and coupled to the robotic assembly 24. The fourth motor is configured to provide rotational motion of the robotic assembly 24.

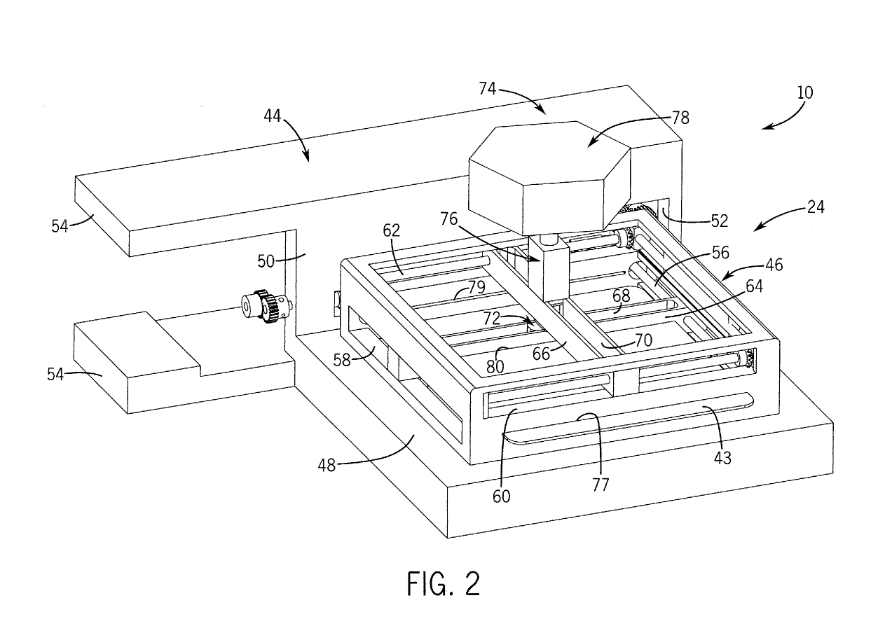

FIG. 2 shows the template system 10 according to one non-limiting example of the present disclosure. As shown in FIG. 2, the template system 10 includes the robotic assembly 24 and a template 43. The robotic assembly 24 is configured to receive and perforate the template 43 in one or more pre-determined locations on the template 43, as will be discussed in detail below.

The robotic assembly 24 includes a base 44 and a robotic assembly frame 46 coupled to the base 44. The base 44 includes an assembly platform 48 that extends substantially perpendicularly from a vertical wall 50. The robotic assembly frame 46 is mounted on the assembly platform 48 of the base 44. The vertical wall 50 includes a plurality of apertures 52 and a pair of opposing horizontal walls 54. The plurality of apertures 52 extend through the vertical wall 50. The pair of opposing horizontal walls 54 extend substantially perpendicularly from the vertical wall 50 in an opposite direction as the assembly platform 48. The pair of opposing horizontal walls 54 and the vertical wall 50 define a cavity where, in one non-limiting example, the first motor 22 and the second motor 30 can be mounted.

The illustrated robotic assembly frame 46 defines a substantially rectangular shape. In other non-limiting examples, the robotic assembly frame 46 may define an alternative shape, as desired. The robotic assembly frame 46 includes a first frame slot 56, a second frame slot 58, a third frame slot 60, and a fourth frame slot 62. The first frame slot 56 slidably receives a one end of a first guide 64 and the second frame slot 58 slidably receives the other end of the first guide 64. The third frame slot 60 slidably receives one end of a second guide 66 and the fourth frame slot 62 slidably receives the other end of the second guide 66. The first guide 64 and the second guide 66 are configured to be arranged substantially perpendicularly to each other and moveable in perpendicular directions with respect to each other. That is, the first hole perforator guide 64 is configured to be movable along the first frame slot 56 and the second frame slot 58 in a direction parallel to the first axis in response to movement of the first motor 22. Similarly, the second guide 66 is configured to be moveable along the third frame slot 60 and the fourth frame slot 62 in a direction parallel to the second axis in response to movement of the second motor 30.

With continues reference to FIG. 2, the first guide 64 is arranged below the second guide 66. The first guide 64 includes a first guide slot 68, and the second guide 66 includes a second guide slot 70. The first guide slot 68 and the second guide slot 70 slidably receive a hole guide coupling 72 which couples the first guide 64 and the second guide 66. The hole guide coupling 72 is arranged at an intersection of the first guide 64 and the second guide 66. The hole guide coupling 72 is configured to slide along the first guide slot 68 and/or the second guide slot 70 in response to movement of the first guide 64 and/or the second guide 66. This enables the hole guide coupling 72 to travel to a pre-determined location (e.g., X,Y coordinate location) by moving the first motor 22 and the second motor 30 which thereby moves the first guide 64 and the second guide 66, respectively.

The hole guide coupling 72 is configured to receive a hole perforating assembly 74. The hole perforating assembly 74 includes a hole punch 76 and a handle 78. The illustrated hole perforating assembly 74 enables a user of the template system 10 to manually drill, or punch, a hole in the template 43 in a pre-determined location on the template 43. It should be known that the hole perforating assembly 74 may be configured to produce a hole in the template 43 using another mechanism known in the art, for example, laser machining or water jets. In other non-limiting examples, the hole perforating assembly 74 can be coupled to the third motor 36 and the fourth motor 42 to automate the drilling process with the hole perforating assembly 74.

The robotic assembly frame 46 includes a first frame template slot 77 arranged below the third frame slot 60 and a second frame template slot 79 arranged below the fourth frame slot 62. The first guide 64 includes a first guide template slot 80. The first frame template slot 77, the second frame template slot 79, and the first guide template slot 80 are horizontally aligned and are each configured to slidably receive the template 43. That is, when a user of the template system 10 desires to perforate the template 43 in one or more pre-determined locations, the user can insert the template 43 through the first frame template slot 78 and thereby through first guide template slot 80 towards the second frame template slot 79.

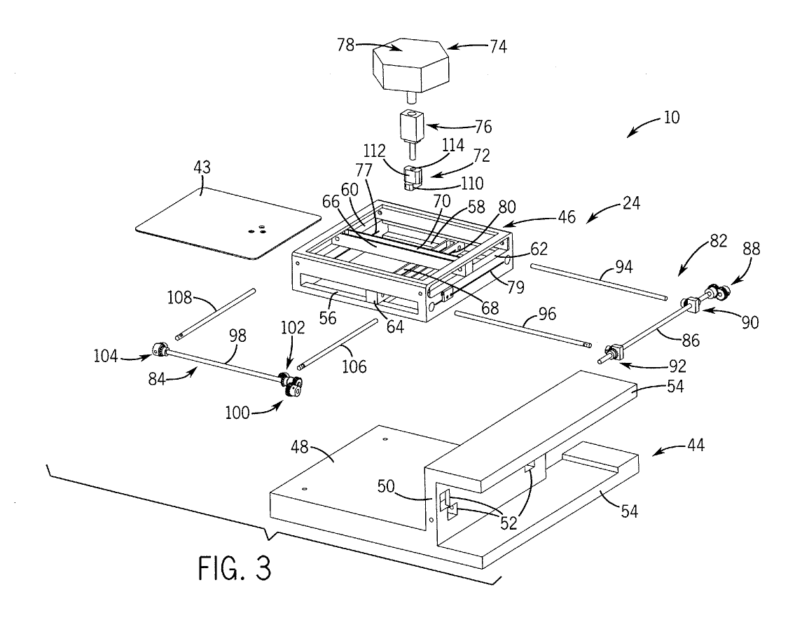

Turning to FIG. 3, the robotic assembly 24 includes a first guide displacement assembly 82 and a second guide displacement assembly 84. The first guide displacement assembly 82 is arranged substantially perpendicularly to the second guide displacement assembly 84. The first guide displacement assembly 82 is coupled to the first guide 64 to convert rotation of the first motor 22 into movement of the first guide 64 along the first axis. The second guide displacement assembly 84 is coupled to the second guide 66 to convert rotation of the second motor 30 into movement of the second guide 66 along the second axis. It is to be understood that the following description of the first guide displacement assembly 82 and the second guide displacement assembly 84 is but one non-limiting example, and other mechanical coupling mechanisms may be used to convert rotation of the motors into movement of the first guide 64 and the second guide 66.

The first guide displacement assembly 80 includes a drive shaft 86, driving gears 88, a first gear set 90, a second gear set 92, a first shaft 94, and a second shaft 96. The first motor 22 is configured to be coupled to the driving gears 88 to rotate the drive shaft 86 in a desired direction. The first gear set 90 is coupled to a first end of the drive shaft 86 and the first shaft 94. The second gear set 92 is coupled to an opposing second end of the drive shaft 86 and the second shaft 96. The first shaft 94 and the second shaft 96 are arranged substantially perpendicularly to the drive shaft 86. The first shaft 94 and the second shaft 96 are each received in a respective coupling aperture 97 of the first guide 64 which couples the first shaft 94 and the second shaft 96 to the first guide 64. The coupling apertures 97 of the first guide 64 are arranged on opposing ends of the first guide 64. In one non-limiting example, the first shaft 94 and the second shaft include external threads.

The second guide displacement assembly 82 includes a drive shaft 98, driving gears 100, a first gear set 102, a second gear set 104, a first shaft 106, and a second shaft 108. The second motor 30 is configured to be coupled to the driving gears 100 to rotate the drive shaft 98 in a desired direction. The first gear set 102 is coupled to a first end of the drive shaft 98 and the first shaft 106. The second gear set 104 is coupled to an opposing second end of the drive shaft 98 and the second shaft 108. The first shaft 106 and the second shaft 108 are arranged substantially perpendicularly to the drive shaft 98. The first shaft 106 and the second shaft 108 are each received in a respective coupling aperture 109 of the second guide 66 which couples the first shaft 106 and the second shaft 108 to the second guide 66. The coupling apertures 109 of the second guide 66 are arranged on opposing ends of the second guide 66. In one non-limiting example, the first shaft 106 and the second shaft 108 include external threads.

In operation, the first motor 22 is configured to rotate the drive shaft 86 in a desired direction. The rotation of the drive shaft 86 in the desired direction results in rotation of the first shaft 94 and the second shaft 96 in desired directions which is converted into movement of the first guide 64 along the first frame slot 56 and the second frame slot 58. As described above, the first frame slot 56 and the second frame slot 58 restrict the first guide 64 to movement along the first axis. The second motor 30 is configured to rotate the drive shaft 98 in a desired direction. The rotation of the drive shaft 98 in the desired direction results in rotation of the first shaft 106 and the second shaft 108 in desired directions which is converted into movement of the second guide 66 along the third frame slot 60 and the fourth frame slot 62. As described above, the third frame slot 60 and the fourth frame slot 62 restrict the second guide 66 to movement along the second axis. As described above, movement of the first guide 64 and/or the second guide 66 results in the hole guide coupling 72 to move along either the first guide slot 68 and/or the second guide slot 70. In this way, the robotic assembly 24 enables the template system 10 to robotically position the guide coupling 72 in a desired position (i.e., X,Y coordinate location) within the robotic assembly frame 46.

With continued reference to FIG. 3, the guide coupling 72 includes a first guide coupling 110, a second guide coupling 112, and a guide coupling aperture 114. The first guide coupling 110 is configured to be received within the first guide slot 68 of the first guide 64. The second guide coupling 112 is arranged above and substantially perpendicularly to the first guide coupling 110 and is configured to be received within the second guide slot 70 of the second guide 66. The guide coupling aperture 114 defines a substantially round shape and extends through the first guide coupling 110 and the second guide coupling 112.

The illustrated template 43 defines a substantially rectangular shape. In other non-limiting examples, the template 43 may define an alternative shape, such as an elliptical shape, a round shape, a square shape, or a polygonal shape, as desired. The template 43 is fabricated from a material that will not interfere with an MRI imaging process. For example, the template 43 can be fabricated from a non-ferrous metal or a plastic material. In most non-limiting examples, the template is sterilized and includes a thin film covering both sides of the template 43 to preserve the sterilized nature of the template 43. It should be known that the illustrated template 43 is shown with a plurality of pre-drilled holes, however, this is merely for illustrative purposes and, in operation, the template 43 would be provided without any pre-drilled holes.

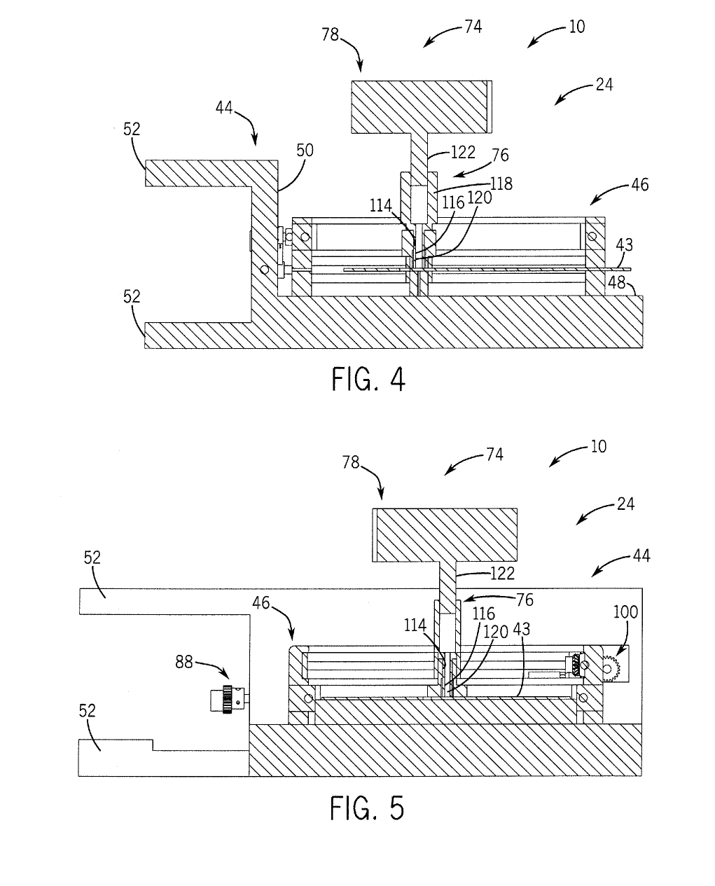

Turning to FIGS. 4 and 5, the hole punch 76 includes a punch tube 116 extending from a punch body 118, and a punch aperture 120. The punch tube 116 is configured to be received within the guide coupling aperture 114 of the guide coupling 72 to facilitate the drilling, or punching, of holes in the template 43. A locating pin 122 is coupled to the handle 78. The locating pin 122 is configured to be received within the punch aperture 120 of the hole punch 76 to enable a user to grip the handle 78 and drill, or punch, a hole in the template 43 by applying a force on the hole punch 76 thereby forcing the punch tube 116 through the template 43.

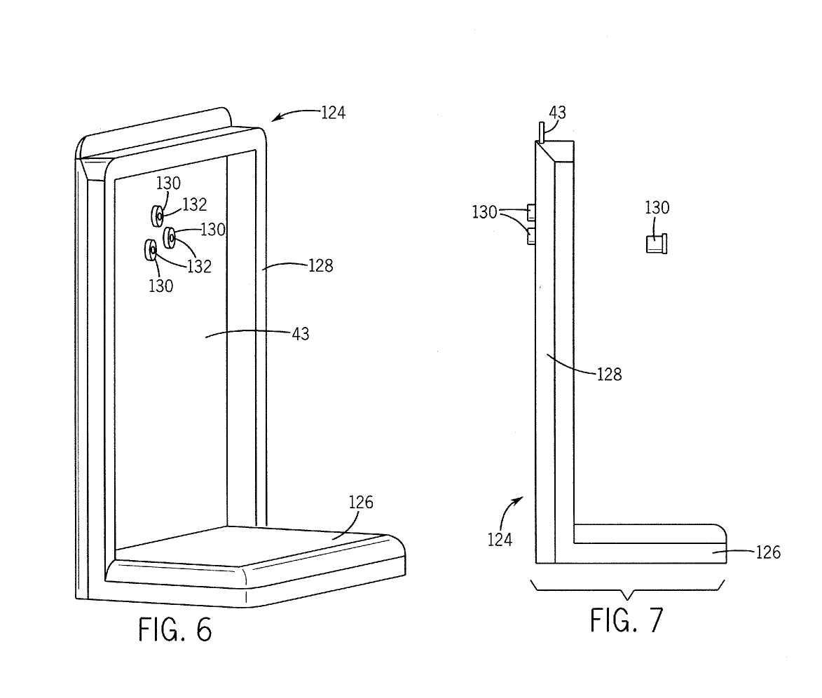

Once the one or more holes in pre-determined locations have been drilled, or punched, into the template 43, the thin film can be removed from the template 43, and the template 43 can be placed in a template holder 124, as shown in FIGS. 6 and 7. The template holder 124 includes a template holder base 126 and a template holder frame 128. The template holder frame 128 extends substantially perpendicularly from the template holder base 126. The template holder 124 includes one or more fiducial markers to enable the location the template holder 124 and thereby the template 43 to be identified relative to a patient after MR imaging. A needle support 130 can be placed into each of the one or more holes drilled, or punched, into the template 43 using the robotic assembly 24. Each of the needle supports 130 include a needle aperture 132 that extends through the needle support 130. The needle supports 130 can provide additional support for a needle which is inserted through the needle aperture 132 when performing a MRI guided medical procedure.

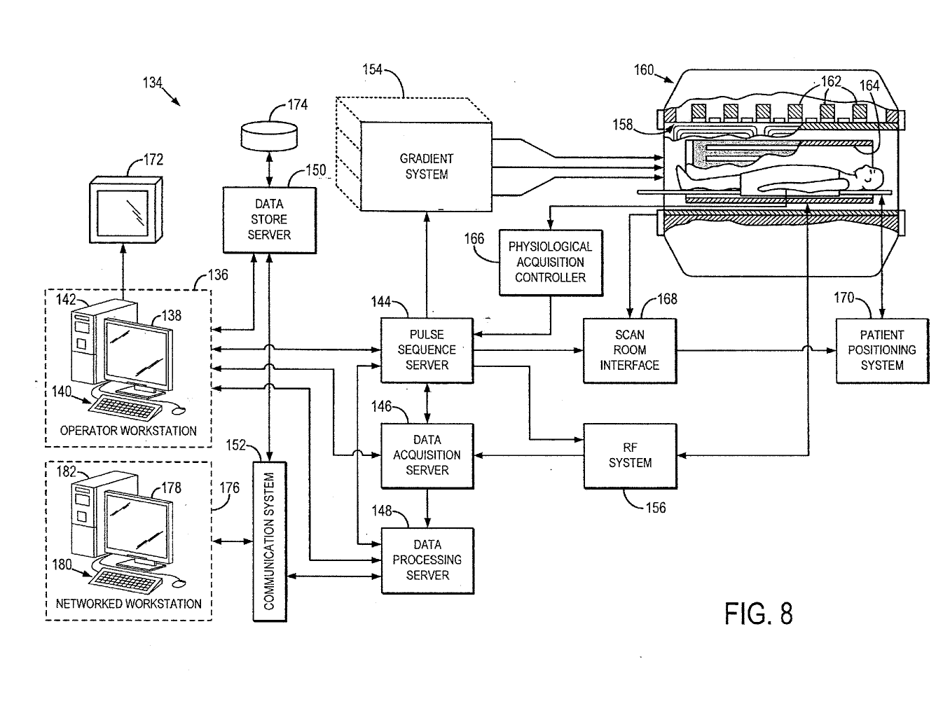

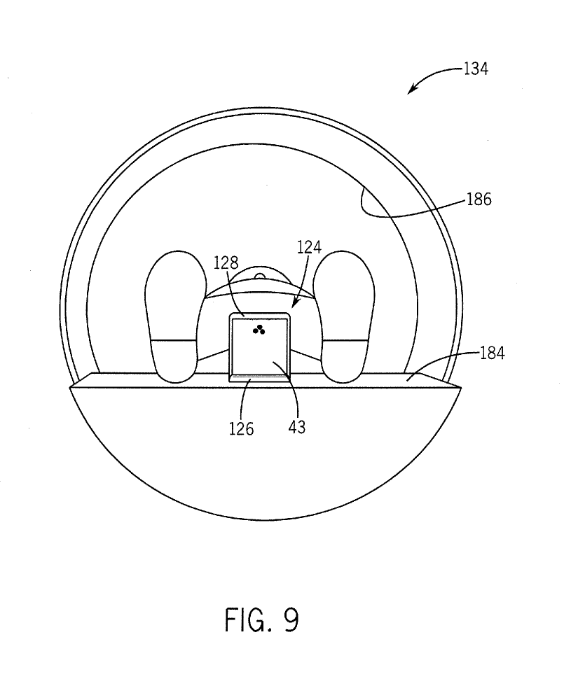

As shown in FIGS. 8 and 9, in one non-limiting example the template system 10 can be used to generate a template 43 for an MRI-guided medical procedure. Referring to FIG. 8, an example of a magnetic resonance imaging (“MRI”) system 134 is illustrated. The MRI system 134 includes an operator workstation 136, which will typically include a display 138; one or more input devices 140, such as a keyboard and mouse; and a processor 142. The processor 142 may include a commercially available programmable machine running a commercially available operating system. The operator workstation 136 provides the operator interface that enables scan prescriptions to be entered into the MRI system 134. In general, the operator workstation 136 may be coupled to four servers: a pulse sequence server 144; a data acquisition server 146; a data processing server 148; and a data store server 150. The operator workstation 136 and each server 144, 146, 148, and 150 are connected to communicate with each other. For example, the servers 144, 146, 148, and 150 may be connected via a communication system 152, which may include any suitable network connection, whether wired, wireless, or a combination of both. As an example, the communication system 152 may include both proprietary or dedicated networks, as well as open networks, such as the internet.

The pulse sequence server 144 functions in response to instructions downloaded from the operator workstation 136 to operate a gradient system 154 and a radiofrequency (“RF”) system 156. Gradient waveforms necessary to perform the prescribed scan are produced and applied to the gradient system 154, which excites gradient coils in an assembly 158 to produce the magnetic field gradients Gx, Gy, and Gz used for position encoding magnetic resonance signals. The gradient coil assembly 158 forms part of a magnet assembly 160 that includes a polarizing magnet 162 and a whole-body RF coil 164 or local RF coil.

In operation, RF waveforms are applied by the RF system 156 to the RF coil 164, or a separate local coil, in order to perform the prescribed magnetic resonance pulse sequence. Responsive magnetic resonance signals detected by the RF coil 164, or a separate local coil, are received by the RF system 156, where they are amplified, demodulated, filtered, and digitized under direction of commands produced by the pulse sequence server 144. The RF system 156 includes an RF transmitter for producing a wide variety of RF pulses used in MRI pulse sequences. The RF transmitter is responsive to the scan prescription and direction from the pulse sequence server 144 to produce RF pulses of the desired frequency, phase, and pulse amplitude waveform. The generated RF pulses may be applied to the whole-body RF coil 164 or to one or more local coils or coil arrays (not shown in FIG. 1).

The RF system 156 also includes one or more RF receiver channels. Each RF receiver channel includes an RF preamplifier that amplifies the magnetic resonance signal received by the coil 130 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

M=\sqrt{I²+Q²} (1);

and the phase of the received magnetic resonance signal may also be determined according to the following relationship:

$$ϕ = {tan}^{- 1} ( \frac{Q}{I} ) . \qquad ( 2 )$$

The pulse sequence server 144 also optionally receives patient data from a physiological acquisition controller 166. By way of example, the physiological acquisition controller 166 may receive signals from a number of different sensors connected to the patient, such as electrocardiograph (“ECG”) signals from electrodes, or respiratory signals from a respiratory bellows or other respiratory monitoring device. Such signals are typically used by the pulse sequence server 144 to synchronize, or “gate,” the performance of the scan with the subject's heart beat or respiration.

The pulse sequence server 144 also connects to a scan room interface circuit 168 that receives signals from various sensors associated with the condition of the patient and the magnet system. It is also through the scan room interface circuit 168 that a patient positioning system 170 receives commands to move the patient to desired positions during the scan.

The digitized magnetic resonance signal samples produced by the RF system 156 are received by the data acquisition server 146. The data acquisition server 146 operates in response to instructions downloaded from the operator workstation 136 to receive the real-time magnetic resonance data and provide buffer storage, such that no data is lost by data overrun. In some scans, the data acquisition server 146 does little more than pass the acquired magnetic resonance data to the data processor server 114. However, in scans that require information derived from acquired magnetic resonance data to control the further performance of the scan, the data acquisition server 146 is programmed to produce such information and convey it to the pulse sequence server 144. For example, during prescans, magnetic resonance data is acquired and used to calibrate the pulse sequence performed by the pulse sequence server 144. As another example, navigator signals may be acquired and used to adjust the operating parameters of the RF system 156 or the gradient system 154, or to control the view order in which k-space is sampled. In still another example, the data acquisition server 146 may also be employed to process magnetic resonance signals used to detect the arrival of a contrast agent in a magnetic resonance angiography (“MRA”) scan. By way of example, the data acquisition server 146 acquires magnetic resonance data and processes it in real-time to produce information that is used to control the scan.

The data processing server 148 receives magnetic resonance data from the data acquisition server 146 and processes it in accordance with instructions downloaded from the operator workstation 136. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 148 are conveyed back to the operator workstation 136 where they are stored. Real-time images are stored in a data base memory cache (not shown in FIG. 1), from which they may be output to operator display 138 or a display 172 that is located near the magnet assembly 160 for use by attending physicians. Also, images are stored in a host database on disc storage 174. When such images have been reconstructed and transferred to storage, the data processing server 148 notifies the data store server 150 or the operator workstation 136. The operator workstation 136 may be used by an operator to archive the images or send the images via a network to other facilities.

The MRI system 134 may also include one or more networked workstations 176. By way of example, a networked workstation 176 may include a display 178; one or more input devices 180, such as a keyboard and mouse; and a processor 182. The networked workstation 176 may be located within the same facility as the operator workstation 136, or in a different facility, such as a different healthcare institution or clinic.

The networked workstation 176, whether within the same facility or in a different facility as the operator workstation 136, may gain remote access to the data processing server 148 or data store server 150 via the communication system 152. Accordingly, multiple networked workstations 142 may have access to the data processing server 148 and the data store server 150. In this manner, magnetic resonance data, reconstructed images, or other data may be exchanged between the data processing server 148 or the data store server 150 and the networked workstations 142, such that the data or images may be remotely processed by a networked workstation 176. This data may be exchanged in any suitable format, such as in accordance with the transmission control protocol (“TCP”), the internet protocol (“IP”), or other known or suitable protocols.

In one non-limiting example, the external controller 12 of the template system 10 may be the processor 142 of the operator workstation 136. In another non-limiting example, the external controller 12 of the template system 10 may be the processor 182 of the network workstation 176. In either case, the external controller 12 is configured to relay information to the controller 14 based on the images taken by the MRI system 134, as will be described below.

Turning to FIG. 9, the template holder base 126 can be mounted to a platform 184 of the MRI system 134 and arranged within an MR bore 186 of the MRI system 134. In the illustrated non-limiting example, the template 43 can include one or more holes dilled, or punched, therein. The one or more holes correspond with locations of harmful (e.g., cancerous) tissue of a patient identified using the MRI system 134 and robotically located then drilled using the robotic assembly 24. In the illustrated non-limiting example, the template holder 124 positions the template 43 adjacent to a perineum of the patient to perform an MRI guided prostate biopsy to detect prostate cancer.

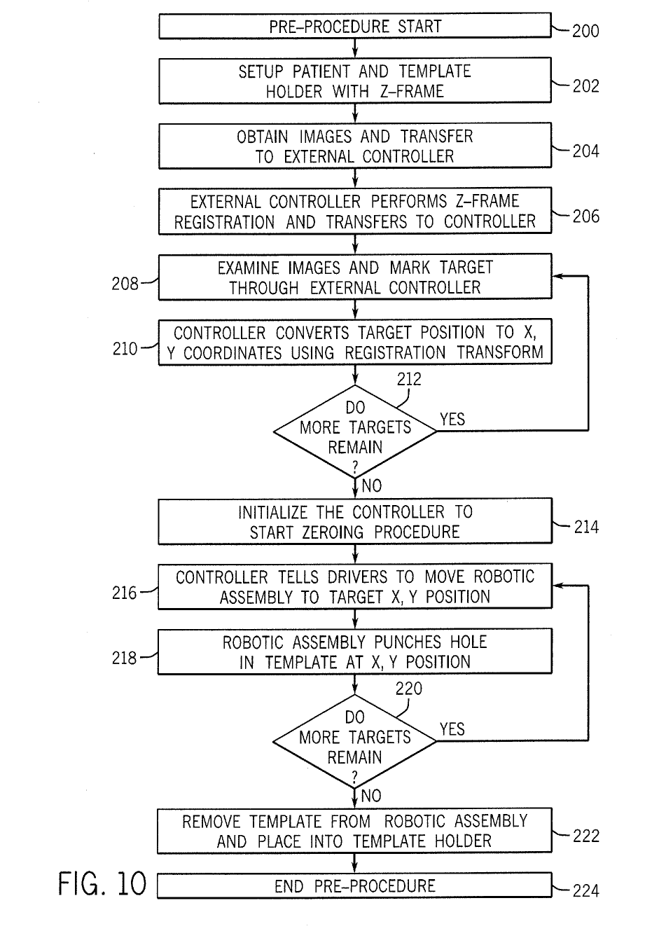

One non-limiting example of the operation of the template system 10 will be described below with reference to FIGS. 1-11. FIG. 10 shows a pre-procedure process (i.e., the steps a user can perform prior to carrying out an image guided medical procedure) for the template system 10. The pre-procedure process starts at step 200 and then the patient and template holder are set up at step 202 by placing the patient within the MR bore 186 and mounting the template holder 124 within the MR bore 186 adjacent to a region of interest on the patient such that the template holder 124 restricts access to the region of interest. Once the patient and template holder are setup at step 202, images of the patient and the template holder are obtained at step 204 using the MRI system 134. As described above, the template holder 124 includes fiducial markers and is therefore viewable in the images obtained at step 204. Once the images are obtained at step 204, the external controller 12 (e.g., the processor 142 of the operator workstation 136) performs a z-frame registration and transfers the registration information to the controller 14. The registration performed at step 206 can correlate the locations defined within the template holder 124 in the obtained images to locations within the robotic assembly 24. Following the registration performed at step 206, a user, or trained medical professional, can then examine at step 208 the images obtained at step 204 and indentify, using the external controller 12, a target area within the region of interest that may contain potentially harmful tissue (e.g., cancerous tissue). For example, by viewing the display 138 of the operator workstation 136 and marking locations using the processor 142. When the target area is identified at step 208, the external controller 12 can communicate the target area to the controller 14 and the controller 14 is configured to convert the position of the target area to X,Y coordinates on a template 43 (i.e., a new template 43 without any holes) within the robotic assembly 24 at step 210 using the registration performed at step 206. It should be understood that although the above described imaging system is in the form of the MRI system 134, other imaging modalities may be used to obtain the necessary images of the patient.

The user, or trained medical professional, then determines at step 212 if additional targets remain in the images obtained at step 204. If additional target areas are identified, the user, or trained medical professional can revert back to step 208 and mark these additional target areas. If no more suspicious areas are identified, the controller 14 can be initialized to start a zeroing procedure at step 214. During the zeroing procedure at step 214, the controller 14 instructs the first driver 16 and the second driver 18 to move the first motor 22 and the second motor 30 to a known zeroed X,Y location. Following the zeroing procedure at step 214, the controller 14 is configured robotically instruct at step 216, via the first driver 16 and the second driver 18, the first motor 22 and the second motor 30 to move the first guide 64 and the second guide 66 such that the guide coupling aperture 114 of the hole guide coupling 72 is in the X,Y coordinates on the template 43 that correspond with the target location(s) marked at step 208. Once the guide coupling aperture 114 of the hole guide coupling 72 is positioned in the correct X,Y coordinate position, a hole can be drilled, or punched, into the template 43 using the hole perforating assembly 74 at step 218. Once the hole is punched in the template 43 at step 218, the controller 14 determines is additional target locations remain. If additional target locations remain, the controller 14 instructs the first motor 22 and the second motor 30 to move to the X,Y coordinates on the template 43 of the next target location at step 216, as described above. If all the target locations have been punched in the template 43, then the thin film can be removed from the template 43 and the template 43 is removed from the robotic assembly 24 and placed in the template holder 124 at step 222 thereby ending the pre-procedure at step 224. It should be known that although the above described pre-procedure of FIG. 10 was described for an X,Y coordinate location, in other non-limiting examples, the robotic assembly can be configured to control, via the third motor 36 and the fourth motor 42, the motion of the hole perforation assembly 74 in a z-direction and/or rotation of the hole perforation assembly 74.

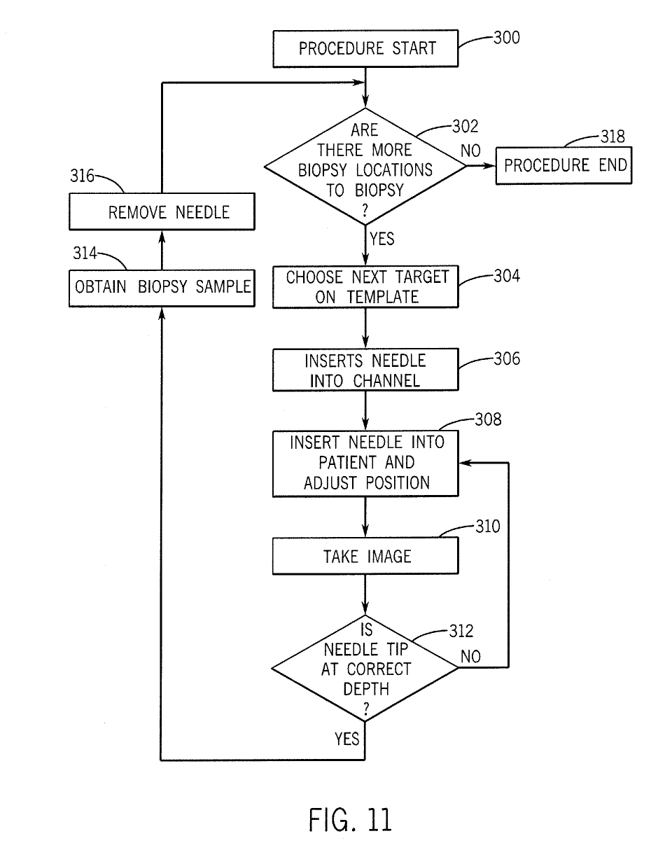

Once the pre-procedure process of FIG. 10 is complete, a user, or trained medical processional, has placed the template 43 having holes in pre-determined locations that were identified as potentially containing suspicious tissue from images of the patient. This enables an image guided medical procedure to then be performed on the patient. FIG. 11 shows the steps for one non-limiting example of an image guided medical procedure in the form of a biopsy procedure. For example, the template 43 created during the pre-procedure of FIG. 10 may be placed adjacent to the perineum of the patient, as shown in FIG. 8, for a prostate biopsy procedure.

As shown in FIG. 11, the procedure starts at step 300 and then it is determined at step 302 if there are more locations on the template 43 to biopsy at step 302. If so, a needle is positioned to be placed within the next hole in the template 43 at step 304. In one non-limiting example, the processor 142 of the MRI system 134 can be configured to instruct the operator display 138 to display the holes in the template 43 for each step of needle insertions. This can enable the users to visualize the specific target hole and sequentially track which holes have been sampled. Once the needle is positioned to be placed within the next hole at step 304, then the needle is inserted through that hole in the template 43 (which was previously identified as a suspicious area) at step 306. The needle can be inserted into the channel manually or automatically. The needle is then inserted into the patient and its position is adjusted at step 308. Once the needle has been inserted into the patient at step 308, an image is obtained at step 310 using the MRI system 134. MRI images are one non-limiting example of the type of image that could be obtained. From the image obtained, it is determined at step 312 if the needle is at a correct depth into the patient (e.g., by viewing the image on the display 138 of the operator workstation 136). If the needle is not at the correct depth, the procedure returns to step 308 where the position of the needle is adjusted. Otherwise, if it is determined at step 312 that the needle is at the correct depth, a biopsy sample is obtained at step 314 and subsequently the needle is removed from the patient at 316. The removal of the needle can be done manually or automatically. If there are more biopsy locations to biopsy, the above described steps are repeated until all of the pre-determined locations (i.e., holes in the template 43) have been biopsied. If there are no more biopsy locations to biopsy, the procedure ends at step 318. Following the end of the procedure, the template 43 can be removed from the template holder 124 and disposed. It should be appreciated that the above described steps may be performed using multiple needles which would enables multiple biopsy samples to be obtained simultaneously.

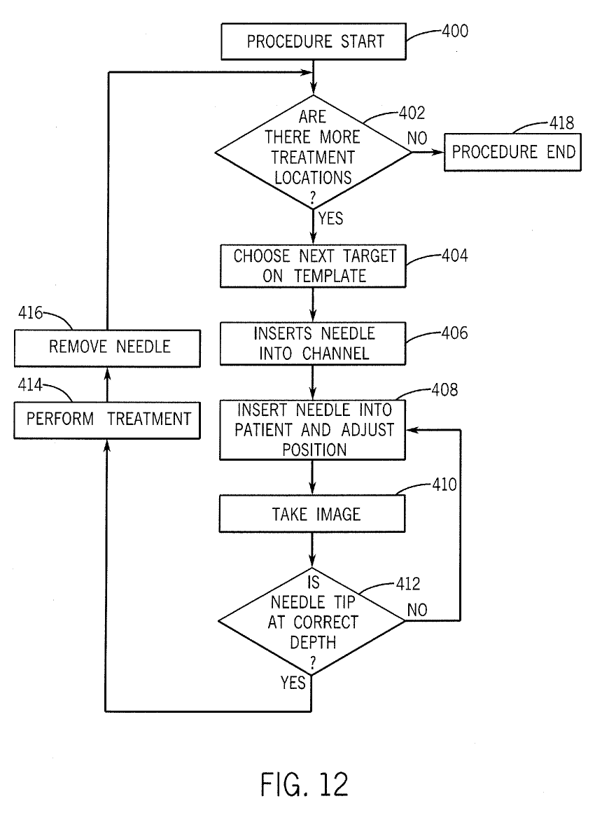

FIG. 12 shows the steps for another non-limiting example of an image guided medical procedure that may be performed following the pre-procedure of FIG. 10. For example, the template 43 created during the pre-procedure of FIG. 10 may be placed adjacent to the patient for an ablation or brachytherapy treatment procedure. As shown in FIG. 12, the treatment procedure steps are similar to the biopsy steps of FIG. 11 with like steps identified using similar reference numbers in the 400's. However, in practice, instead of a biopsy sample at step 314, a treatment is performed at step 414.

As described above, the present disclosure provides a template system 10 which can be used to robotically locate areas of interest within a patient to more accurately perform an image guided medical procedure. This can enable non discrete guidance to the identified locations for higher accuracy and result is less failed procedures.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

We claim:

1. A method of creating a template, for an image guided medical procedure, using a robotic assembly of a template system, the template system including a template holder configured to receive the template, a guide coupling aperture, and a controller in communication with an imaging system, the method comprising:
arranging the template holder adjacent to a region of interest of a patient such that the template holder restricts access to the region of interest of the patient;
acquiring one or more images of the template holder and the region of interest of the patient with the imaging system;
determining, using the one or more images and the controller, a geometric location of the region of interest;
engaging the template with the robotic assembly of the robotic-guided assembly system;
robotically guiding the guide coupling aperture to a location on the template corresponding to the geometric location of the region of interest; and
creating a hole in the template at the location on the template corresponding to the geometric location of the region of interest.

2. The method of claim 1 wherein robotically guiding the guide coupling aperture to a location on the template corresponding to the geometric location of the region of interest comprises:
instructing, via the controller of the template system, a first motor to move a first guide coupled to the guide coupling aperture along a first axis and/or a second motor to move a second guide coupled to the guide coupling aperture along a second axis.

3. The method of claim 1 further comprising, upon creating the hole in the template, disengaging the template from the robotic assembly; and engaging the template with the template holder.

4. The method of claim 3 further comprising inserting a needle support into the hole in the template.

5. The method of claim 3 further comprising upon disengaging the template from the robotic assembly, removing a protective thin film from both sides of the template.

6. The method of claim 1, wherein the imaging system is an MRI system.

7. A method of performing an image guide medical procedure on a patient within an imaging system, the method comprising:
placing a template holder adjacent to a region of interest of the patient such that the template holder restricts access to the region of interest of the patient;
robotically creating a template by:
capturing one or more images of the template holder and the region of interest of the patient with the imaging system;
identifying, using the one or more images, an area of tissue within the region of interest of the patient;
engaging the template with a robotic assembly of a robotic-guided assembly system;
robotically guiding a guide coupling aperture of the robotic assembly to a location on the template corresponding to the geometric location of the identified area of tissue;
creating a hole in the template at the location on the template corresponding to the geometric location of the identified area of tissue;
upon creating the hole in the template, disengaging the template from the robotic assembly and engaging the template with the template holder;
inserting a needle through the hole in the template and into the patient;
verifying a position of the needle within the patient via the imaging system;
performing a treatment on the identified area of tissue within the patient; and
removing the needle from the patient.

8. The method of claim 7 wherein robotically guiding the guide coupling aperture to a location on the template corresponding to the geometric location of the identified area of tissue comprises:
instructing, via a controller of the template system, a first motor to move a first guide coupled to the guide coupling aperture along a first axis and/or a second motor to move a second guide coupled to the guide coupling aperture along a second axis.

9. The method of claim 7 further comprising inserting a needle support into the hole drilled in the template.

10. The method of claim 7 further comprising upon disengaging the template from the robotic assembly, removing a protective thin film from both sides of the template.

11. The method of claim 7, wherein the imaging system is an MRI system.

12. A method for automated guidance of an image-guided medical procedure, the method comprising:
accessing one or more images of a region of interest of a patient and a template holder arranged adjacent to the region of interest of the patient such that the template holder restricts access to the region of interest of the patient from at least one direction;
using the one or more images of the region of interest and template holder, identifying desired tissue in the region of interest and a geometric location relative to the template holder that provides access to the desired tissue through the template holder from the at least one direction;
communicating the geometric location to a controller configured to create a hole in a template configured to be coupled with the template holder to provide access to the desired tissue from the at least one direction through the template when the template is engaged with the template holder;
accessing one or more images of a needle being inserted through the hole in the template from the at least one direction and into the patient toward the desired tissue; and
verifying a position of the needle within the patient using the one or more images of the needle being inserted.

13. The method of claim 12, wherein the imaging system is an MRI system.

14. A template system for an image guided medical procedure performed on a patient, the template system comprising:
a first motor;
a second motor;
a robotic assembly including:
a robotic assembly frame including a first guide and a second guide, the first guide moveable along a first axis and operably coupled to the first motor, the second guide moveable along a second axis substantially perpendicular to the first axis and operably coupled to the first motor;
a hole guide coupling to couple the first guide to the second guide and including a guide coupling aperture;
a template configured to be received within the robotic assembly frame; and
a controller configured to control the first motor and the second motor to move the first guide and the second guide and thereby position the guide coupling aperture in one or more pre-determined locations.

15. The template system of claim 14 further comprising an external controller configured to communicate the one or more pre-determined locations to the controller based on one or more images of the patient captured with an imaging system.

16. The template system of claim 15, wherein the one or more pre-determined locations correspond with one or more areas of abnormal tissue within the patient identified in the one or more images captured by the imaging system.

17. The template system of claim 15, wherein the imaging system is an MRI system.

18. The template system of claim 14 further comprising a first guide displacement assembly coupled to the first guide and the first motor to convert motion of the first motor into movement of the first guide, and a second guide displacement assembly coupled to the second guide and the second motor to convert motion of the second motor into movement of the second guide.

19. The template system of claim 14 further including a hole perforating assembly configured to be received within the guide coupling aperture.

20. The template system of claim 19, wherein the hole perforating assembly includes a hole punch and a handle.

21. The template system of claim 20, wherein the hole punch includes a punch body and a punch tube extending from the punch body, the punch tube configured to be received within the guide coupling aperture.

22. The template system of claim 14 further comprising a template holder configured to receive the template.

23. The template system of claim 22, wherein the template holder includes one or more fiducial markers to enable the template holder to be located by an imaging system.

24. The robotic guide template system of claim 22, wherein the template holder is fabricated from a non-ferrous material.

25. The template system of claim 17, wherein the template is fabricated from a non-ferrous material.
